(19) United States
(12) Patent Application Publication
(10) Pub. No.: US 2014/0025116 A1
(43) Pub. Date: Jan. 23, 2014

(54) Fixing Structure of Bone Screws and a Connecting Rod for a Minimally Invasive Surgery

(71) Applicant: Chih-Hsuan Wei, Chiayi City (TW)

(72) Inventor: Chih-Hsuan Wei, Chiayi City (TW)

(21) Appl. No.: 13/555,677

(22) Filed: Jul. 23, 2012

(51) Int. Cl. A61B 17/70

(52) U.S. Cl. 606/255

(57) ABSTRACT

A fixing structure of bone screws and a connecting rod for a minimally invasive surgery contains: a rigid sleeve having a receiving space, two slots, and a through hole, the receiving space and the through hole being defined on two ends of the rigid sleeve, and the two slots being formed on two opposite sides of an outer wall of the rigid sleeve and communicate with the receiving space; a flexible column being circular and retained in the receiving space of the rigid sleeve. Thereby, a complete rigid limitation and partial flexible limitations generate between at least one bone screw, hence only a tiny opening is cut in the minimally invasive surgery to facilitate surgery recovery and to lower infection risk.

### BACKGROUND OF THE INVENTION

1. Field of the Invention

The present invention relates to a fixing structure of bone screws and a connecting rod for a minimally invasive surgery which allows fixing a rigid sleeve and a flexible column to generate a complete rigid limitation and partial flexible limitations.

2. Description of the Prior Art

A conventional fixing structure of bone screws and a connecting rod is made of rigid material, so between the bone screws lacks a flexible bending and rotating ability, and the spine can not bend or rotate flexibly, thus limiting applicable options in treatment and corrective rehabilitations.

Although the convention fixing structure of the bone screws and the connecting rod can lock and position the spines, the bone screws limit the flexibility in a movement and a rotation. Besides, the rigid sleeve can not match with the flexible column well.

The present invention has arisen to mitigate and/or obviate the afore-described disadvantages.

### SUMMARY OF THE INVENTION

The primary object of the present invention is to provide a fixing structure of bone screws and a connecting rod for a minimally invasive surgery which allows fixing a rigid sleeve and a flexible column to generate a complete rigid limitation and partial flexible limitations, thus providing applicable options to different treatments and corrective rehabilitations.

Another object of the present invention is to provide a fixing structure of bone screws and a connecting rod for a minimally invasive surgery which simplifies a connection of the rigid sleeve and the flexible column and obtains an engagement and positioning between a plurality of spines.

A fixing structure of bone screws and a connecting rod for a minimally invasive surgery in accordance with a preferred embodiment of the present invention contains:

a rigid sleeve having a receiving space, two slots, and a through hole, the receiving space and the through hole being defined on two ends of the rigid sleeve, and the two slots being formed on two opposite sides of an outer wall of the rigid sleeve and communicate with the receiving space;

a flexible column being circular and retained in the receiving space of the rigid sleeve.

Thereby, the rigid sleeve limits a movement of the plurality of spines via the bone screws.

The flexible column allows the bone screws moving with the plurality of spines.

The rigid sleeve matches with the flexible column to increase applicable flexibility.

The rigid sleeve is connected with the flexible column easily to save operation time.

The rigid sleeve is simplified to control design and production cost.

The flexible column is made of various flexible materials, so optional materials are diversified without being limited.

### BRIEF DESCRIPTION OF THE DRAWINGS

FIG. 1 is a perspective view showing the exploded components of a fixing structure of bone screws and a connecting rod for a minimally invasive surgery according to a preferred embodiment of the present invention.

FIG. 2 is a perspective view showing the assembly of the fixing structure of the bone screws and the connecting rod for the minimally invasive surgery according to the preferred embodiment of the present invention.

FIG. 3 is a cross sectional view showing the assembly of the fixing structure of the bone screws and the connecting rod for the minimally invasive surgery according to the preferred embodiment of the present invention.

FIG. 4 is a perspective view showing the operation of the fixing structure of the bone screws and the connecting rod for the minimally invasive surgery according to the preferred embodiment of the present invention.

FIG. 5 is a cross sectional view showing the application of the fixing structure of the bone screws and the connecting rod for the minimally invasive surgery according to the preferred embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

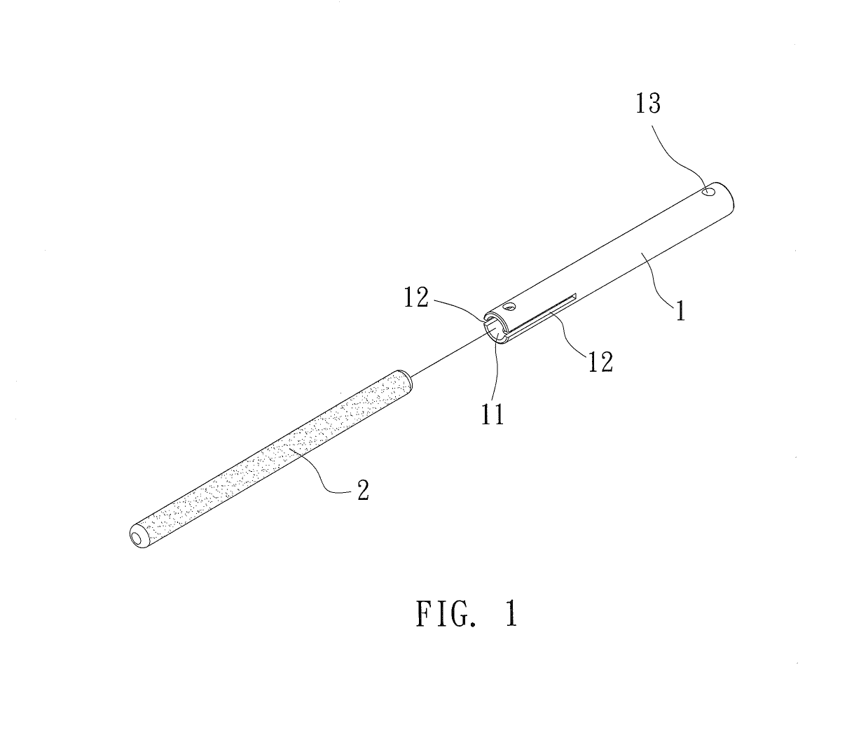

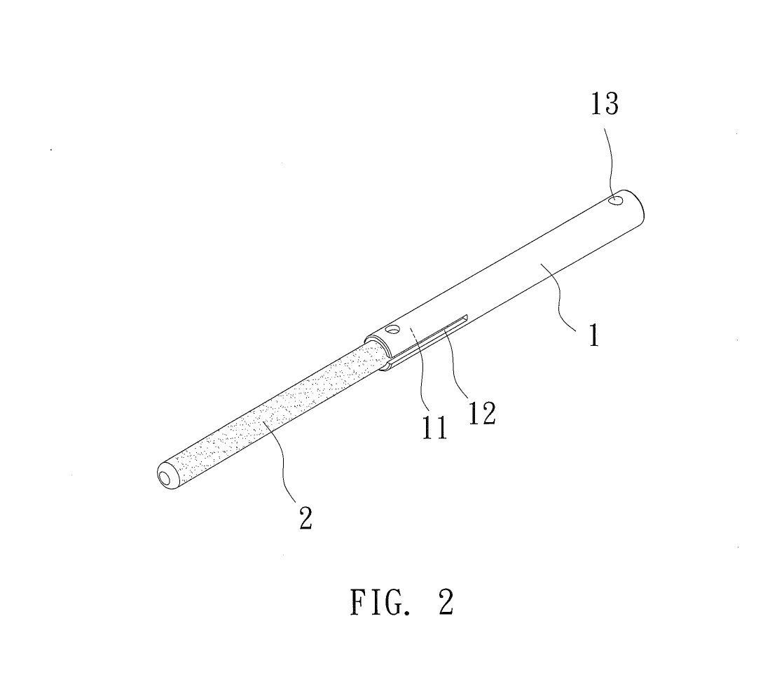

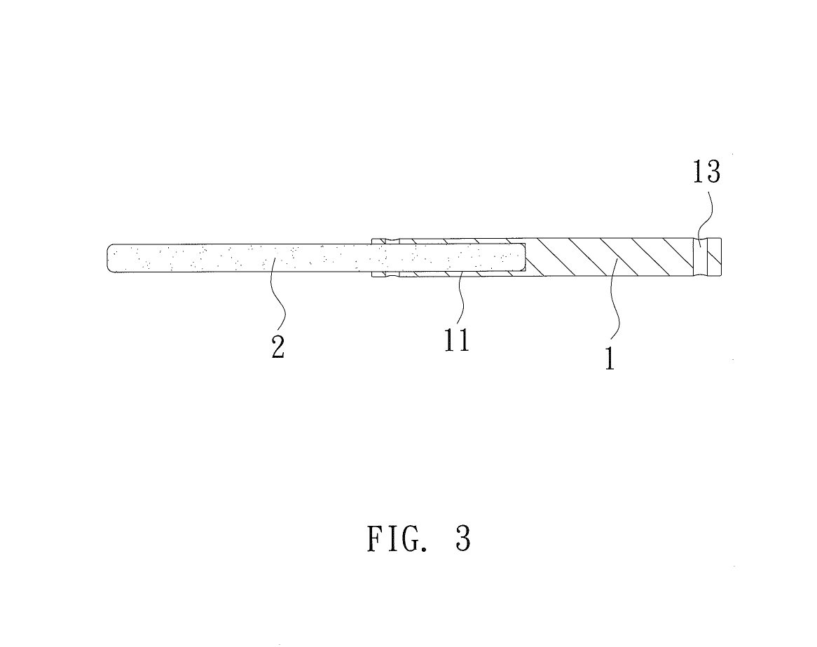

Referring to FIGS. 1-3, a fixing structure of bone screws and a connecting rod for a minimally invasive surgery according to a preferred embodiment of the present invention comprises:

a rigid sleeve 1 having a receiving space 11, two slots 12, and a through hole 13, wherein the receiving space 11 and the through hole 13 are defined on two ends of the rigid sleeve 1, and the two slots 12 are formed on two opposite sides of an outer wall of the rigid sleeve 1 and communicate with the receiving space 11, the rigid sleeve 1 is made of metal material;

a flexible column 2 being circular and retained in the receiving space 11 of the rigid sleeve 1 and being made of plastic, rubber, high-density foam, or silicone.

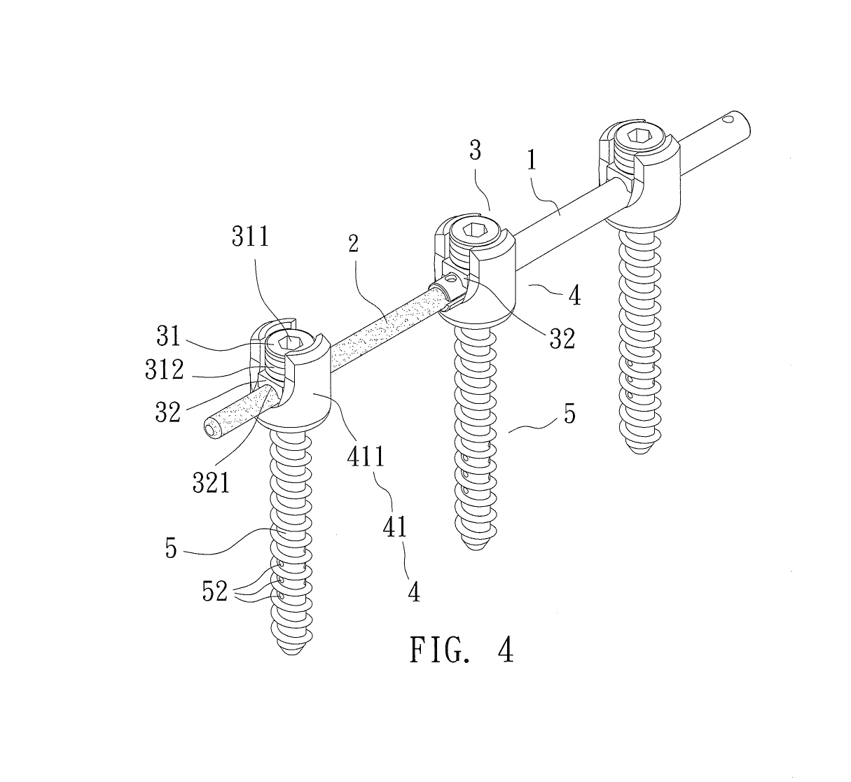

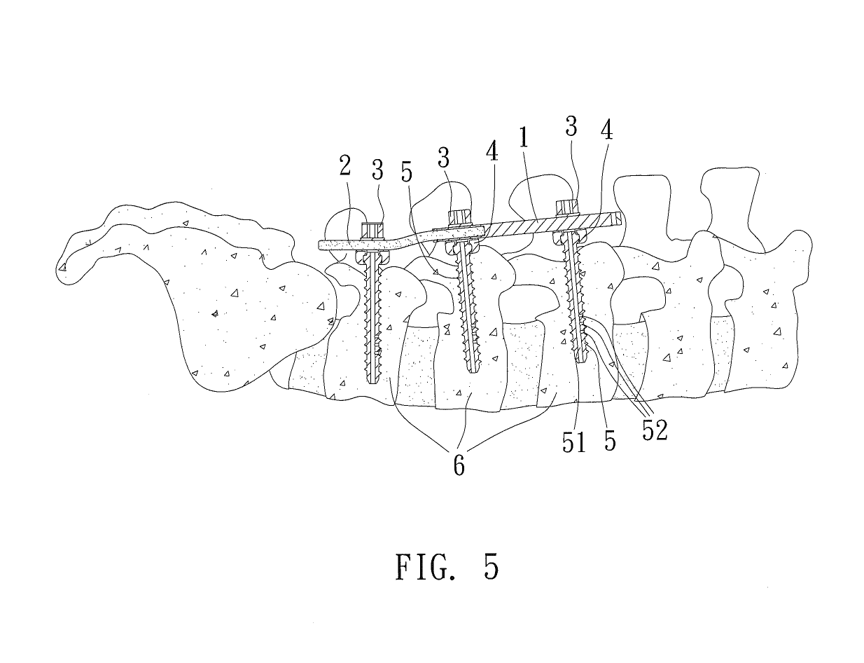

Referring further to FIG. 4, the fixing structure of the present invention also comprises a plurality of retaining members 3, each having a locking element 31. The locking element 31 has an inner hexagonal orifice 311 defined on a top end thereof so that a tool (such as a hexagon wrench) is retained with the inner hexagonal orifice 311 to rotate the locking member 31, such that outer threads 312 of the locking element 31 screw in a body 4 of at least one bone screw and between two external fences 411 of at least one hold 41, the rigid sleeve 1 and the flexible column 2 are retained by at least one engaging seat 32 to obtain a stable engagement and positioning. Furthermore, at least one screw 5 includes a plurality of inlets 52 and is locked on at least one spine 6 as illustrated in FIG. 5.

It is to be noted that as between two bodies 4 is connected the rigid sleeve 1, the two bodies 4 do not move relative to each other, and as between another two bodies 4 is connected the flexible column 2, the another two bodies 4 move relative to each other. In other words, in a gap between two fixed spines 6 (as shown in FIG. 5), the rigid sleeve 1 is connected with two bodies 4 of two bone screws in the two fixed spines 6, and in another gap between two movable spines 6 (as shown in FIG. 5), the flexible column 2 of the rigid sleeve 1 is connected with two bodies 4 of two bone screws in the two movable spines 6. In another embodiment, when desiring to fix a joint of the spine but to make the joint bend and move as shown in FIG. 5, the bone screw in a middle spine 6 connects with a portion of the flexible column 2 which is fitted into the rigid sleeve 1.

As illustrated in FIG. 5, in the minimally invasive surgery, the at least one screw 5 is screwed on the at least one spine 6, and each of the plurality of the retaining members 3 is retained with the at least one body 4 of the at least one bone screw, and then the rigid sleeve 1 is in connection with the flexible column 2 so that a complete rigid limitation and partial flexible limitations generate between the at least one bone screw, hence only a tiny opening is cut in the minimally invasive surgery to facilitate surgery recovery and to lower infection risk. In addition, an inner aperture 51 of each screw 5 communicates with each spine 6 via the plurality of inlets 52 so as to fill a bulking agent.

While we have shown and described various embodiments in accordance with the present invention, it is clear to those skilled in the art that further embodiments may be made without departing from the scope of the present invention.

What is claimed is:

1. A fixing structure of bone screws and a connecting rod for a minimally invasive surgery comprises:
a rigid sleeve having a receiving space, two slots, and a through hole, the receiving space and the through hole being defined on two ends of the rigid sleeve, and the two slots being formed on two opposite sides of an outer wall of the rigid sleeve and communicate with the receiving space;
a flexible column being circular and retained in the receiving space of the rigid sleeve.

2. The fixing structure of the bone screws and the connecting rod for the minimally invasive surgery as claimed in claim 1, wherein the rigid sleeve is made of metal material.

3. The fixing structure of the bone screws and the connecting rod for the minimally invasive surgery as claimed in claim 1, wherein the flexible column is made of plastic.

4. The fixing structure of the bone screws and the connecting rod for the minimally invasive surgery as claimed in claim 1, wherein the flexible column is made of rubber.

5. The fixing structure of the bone screws and the connecting rod for the minimally invasive surgery as claimed in claim 1, wherein the flexible column is made of high-density foam.

6. The fixing structure of the bone screws and the connecting rod for the minimally invasive surgery as claimed in claim 1, wherein the flexible column is made of silicone.
